(19) United States
(12) Patent Application Publication
(10) Pub. No.: US 2023/0000785 A1
(43) Pub. Date: Jan. 5, 2023

(54) CROSSLINKED PLA-BASED COPOLMERS

(71) Applicant: Pendant Biosciences, Inc., Nashville, TN (US)

(72) Inventors: Carl Eric Elmquist, Franklin, TN (US); Timothy Tordella Ruckh, Mountain View, CA (US)

(21) Appl. No.: 17/843,137

(22) Filed: Jun. 17, 2022

Related U.S. Application Data
(63) Continuation of application No. PCT/US2020/066347, filed on Dec. 21, 2020.
(60) Provisional application No. 62/950,281, filed on Dec. 19, 2019.

(51) Int. Cl. A61K 9/51; A61K 9/50; A61K 9/16

(52) U.S. Cl. CPC A61K 9/5192; A61K 9/5089; A61K 9/1682; A61K 9/5153; A61K 9/5031; A61K 9/1647; B82Y 5/00

(57) ABSTRACT

Embodiments described herein relate generally to compounds comprising allyl lactide residues. One aspect described herein relates generally to a compound or a pharmaceutically acceptable salt thereof, comprising allyl lactide residues and lactide residues, wherein the compound or pharmaceutically acceptable salt thereof is substantially free of valerolactone residues. Another aspect relates to a method of incorporating a drug into a compound, comprising:
(i) providing a compound or a pharmaceutically acceptable salt thereof, comprising allyl lactide residues and lactide residues, wherein the compound or pharmaceutically acceptable salt thereof is substantially free of valerolactone residues;
(ii) incubating the compound and a drug in the presence of a solvent for an incubation period to form a drug-loaded compound; and
(iii) separating the drug-loaded compound from the solvent.

### RELATED APPLICATIONS

This application is a continuation of International Patent Application No. PCT/2020/066347, filed Dec. 21, 2020 and entitled, “CROSSLINKED PLA-BASED COPOLYMERS,” which claims priority and benefit of U.S. Provisional Application No. 62/950,281, filed Dec. 19, 2019 and entitled “CROSSLINKED PLA-BASED COPOLYMERS,” the disclosures of which are hereby incorporated by reference in their entireties.

### FIELD OF THE DISCLOSURE

The present disclosure is directed to compounds comprising allyl lactide residues, lactide residues, and substantially no valerolactone residues.

### BACKGROUND

Control of drug release rate is important, in order to ensure a proper level of drug exposure to a patient. To achieve adequate drug exposure many drugs require administration daily or more frequently. However, with such frequent administration requirements, any number of circumstances can affect patient compliance and required frequency of consumption. If the drug is delivered via a slow-release mechanism, this can reduce the uncertainty associated with drug delivery to the patient and reduce the burden of responsibility on the patient. However, current technology is limited with respect to control of drug release rate. Polymer matrices are not sufficiently tunable to release a drug in vivo with desired levels of speed control. Therefore, there exists a need for new methods and mechanisms of drug release.

### SUMMARY

The technology disclosed herein enables tunable release of drugs from a compound (e.g., a polymer matrix comprising allyl lactide, lactide, and optionally glycolide residues). The release rate of drugs can be controlled by crosslinking density. In some embodiments, the release rate of drugs can be controlled by the molecular weight of the compound.

Embodiments described herein relate generally to compounds comprising allyl lactide residues. A tunable and predictable release rate of a drug can be achieved with the proper compound development, crosslinking density, and residues disclosed herein. For instance, the precision of drug release can be finely tuned for a slow, controlled release.

One aspect described herein relates generally to a compound or a pharmaceutically acceptable salt thereof, comprising allyl lactide residues and lactide residues, wherein the compound or pharmaceutically acceptable salt thereof is substantially free of valerolactone residues.

In some embodiments, the compound can further comprise glycolide residues.

In some embodiments, the compound can include the structure:

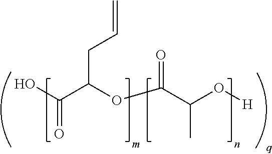

wherein:
m is an integer from about 1 to about 1,000;
n is an integer from about 1 to about 1,000;
q is an integer from about 1 to about 100; and
wherein the compound or pharmaceutically acceptable salt thereof is substantially free of valerolactone residues.

In some embodiments, the compound can include the structure:

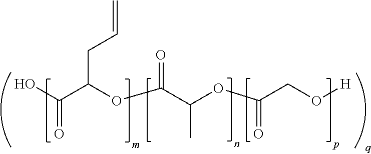

wherein
p is an integer from about 1 to about 1,000;
m is an integer from about 1 to about 100;
n is an integer from about 1 to about 100; and
q is an integer from about 1 to about 100.

In some embodiments, m can be an integer from about 1 to about 25.

In some embodiments, n can be an integer from about 1 to about 25.

In some embodiments, m/(m+n) can be from about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, about 0.9 to about 0.99, about 0.02, about 0.06, about 0.12, about 0.26, or about 0.48.

In some embodiments, m/(m+n+p) can be from about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, about 0.9 to about 0.99, about 0.02, about 0.06, about 0.12, about 0.26, or about 0.48.

In some embodiments, the residues can form a compound that can be crosslinked with a crosslinker.

In some embodiments, the crosslinker can include a thiol moiety. In some embodiments, the crosslinker can be

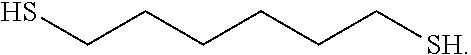

In some embodiments, the compound can be about 10% to about 99% crosslinked, about 1% to about 9% crosslinked, about 10% to about 19% crosslinked, about 20% to about 29% crosslinked, about 30% to about 39% crosslinked, about 40% to about 49% crosslinked, about 50% to about 59% crosslinked, about 60% to about 69% crosslinked, about 70% to about 79% crosslinked, about 80% to about 89% crosslinked, about 90% to about 99% crosslinked, about 2% crosslinked, about 6% crosslinked, about 12% crosslinked, about 26% crosslinked, about 48% crosslinked.

In some embodiments, the compound retains more than 90% of its initial mass after exposure to a phosphate-buffer saline (PBS) for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days at about 37° C.

In some embodiments, the PBS includes about 0.5 wt % sodium dodecyl sulfate (SDS).

In some embodiments, the drug is a small molecule drug.

In some embodiments, the drug is a large molecule drug.

In some embodiments, a macroparticle can be formed, comprising any of the aforementioned compounds.

In some embodiments, a microparticle can be formed, comprising any of the aforementioned compounds.

In some embodiments, a nanoparticle can be formed, comprising any of the aforementioned compounds.

Another aspect relates to a method of incorporating a drug into a compound, comprising:

(i) providing a compound or a pharmaceutically acceptable salt thereof, comprising allyl lactide residues and lactide residues, wherein the compound or pharmaceutically acceptable salt thereof is substantially free of valerolactone residues;
(ii) incubating the compound and a drug in the presence of a solvent for an incubation period to form a drug-loaded compound; and
(iii) separating the drug-loaded compound from the solvent.

In some embodiments, the compound can further include glycolic acid residues.

In some embodiments, the compound can include the structure:

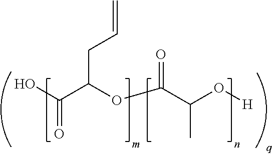

wherein:
m is an integer from about 1 to about 1,000;
n is an integer from about 1 to about 1,000;
q is an integer from about 1 to about 100; and
wherein the compound or pharmaceutically acceptable salt thereof is substantially free of valerolactone residues.

In some embodiments, the compound can include the structure:

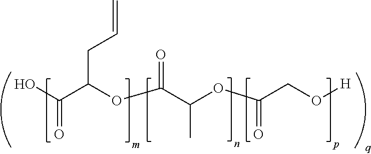

wherein
p is an integer from about 1 to about 1,000;
m is an integer from about 1 to about 100;
n is an integer from about 1 to about 100, and
q is an integer from about 1 to about 100.

In some embodiments, m can be an integer from about 1 to about 25.

In some embodiments, n can be an integer from about 1 to about 25.

In some embodiments, m/(m+n) can be from about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, about 0.9 to about 0.99, about 0.02, about 0.06, about 0.12, about 0.26, or about 0.48.

In some embodiments, m/(m+n+p) can be from about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, about 0.9 to about 0.99, about 0.02, about 0.06, about 0.12, about 0.26, or about 0.48.

In some embodiments, the residues can form a compound that can be crosslinked with a crosslinker.

In some embodiments, the crosslinker can include a thiol moiety. In some embodiments, the crosslinker can be

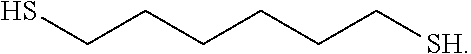

In some embodiments, the compound can be about 10% to about 99% crosslinked, about 1% to about 9% crosslinked, about 10% to about 19% crosslinked, about 20% to about 29% crosslinked, about 30% to about 39% crosslinked, about 40% to about 49% crosslinked, about 50% to about 59% crosslinked, about 60% to about 69% crosslinked, about 70% to about 79% crosslinked, about 80% to about 89% crosslinked, about 90% to about 99% crosslinked, about 2% crosslinked, about 6% crosslinked, about 12% crosslinked, about 26% crosslinked, about 48% crosslinked.

In some embodiments, the compound retains more than 90% of its initial mass after exposure to an aqueous medium for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days at about 37° C.

In some embodiments, the aqueous medium can include phosphate buffer saline (PBS).

In some embodiments, the PBS includes about 0.5 wt % sodium dodecyl sulfate (SDS).

In some embodiments, the drug can be Paclitaxel (PTX), Triamcinolone acetonide (TAA), or Triamcinolone hexacetonide (TAH).

In some embodiments, the method can include forming the drug-loaded compound into a particle.

In some embodiments, the particle can be a nanoparticle.

In some embodiments, the particle can be a microparticle.

In some embodiments, the particle can be a macroparticle.

In some embodiments, the solvent can include a hydrophobic solvent.

In some embodiments, the solvent can include a hydrophilic solvent.

In some embodiments, the solvent can include a hydrophobic solvent and the drug can be a small molecule drug (e.g., PTX, TAH).

In some embodiments, the solvent can include a hydrophobic solvent and the drug can be a large molecule drug (e.g., an antibody).

In some embodiments, the solvent can include a hydrophilic solvent and the drug can be a small molecule drug.

In some embodiments, the solvent can include a hydrophilic solvent and the drug can be a large molecule drug.

In some embodiments, the solvent can include dichloromethane.

In some embodiments, the solvent can include water.

In some embodiments, the solvent can include ethanol.

In some embodiments, the solvent can include methanol.

In some embodiments, the solvent can include acetic acid.

In some embodiments, the solvent can include propanol.

In some embodiments, the solvent can include carbon tetrachloride.

In some embodiments, the solvent can include hexane.

In some embodiments, the solvent can include benzene.

In some embodiments, the solvent can include tetrahydrofuran (THF).

In some embodiments, the solvent can include dimethylsulfoxide (DMSO).

In some embodiments, the solvent can include DMSO and THF in approximately a 50:50 mixture by weight.

In some embodiments, the incubation period can be about 1 day, about 2 days, about 3 days, about 4 days, about 5 days, about 6 days, about 7 days, about 8 days, about 9 days, or about 10 days.

In some embodiments, the drug-loaded compound can include about 30 wt % to about 50 wt % of the drug.

In some embodiments, the cumulative release rate of the drug from the drug-loaded compound in an aqueous medium can be between about 10 wt % and about 20 wt % over a period of 35 days.

In some embodiments, the aqueous medium can be a solution of PBS and 0.5% SDS.

In some embodiments, the cumulative release rate of the drug from the drug-loaded compound in an aqueous medium can be between about 40 wt % and about 50 wt % over a period of 60 days.

In some embodiments, the aqueous medium can be a solution of PBS and 0.5% SDS.

In some embodiments, the drug-loaded compound releases no more than about 10%, no more than about 20%, no more than about 30%, no more than about 40%, no more than about 50%, no more than about 60%, or no more than about 70% of the drug after 30 days.

Another aspect relates to a drug-loaded compound or pharmaceutically acceptable salt thereof, comprising allyl lactide residues and lactide residues, wherein the drug-loaded compound or pharmaceutically acceptable salt thereof is substantially free of valerolactone residues.

In some embodiments, the drug-loaded compound can include glycolide residues.

In some embodiments, the drug-loaded compound can include the structure:

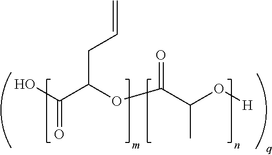

wherein:
m is an integer from about 1 to about 1,000;
n is an integer from about 1 to about 1,000;
q is an integer from about 1 to about 100; and
wherein the drug-loaded compound or pharmaceutically acceptable salt thereof is substantially free of valerolactone residues.

In some embodiments, the drug-loaded compound can include the structure:

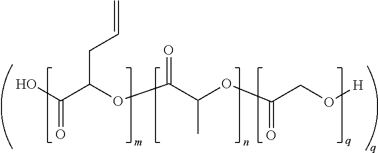

wherein
p is an integer from about 1 to about 1,000;
m is an integer from about 1 to about 100;
n is an integer from about 1 to about 100; and
q is an integer from about 1 to about 100.

In some embodiments, m can be an integer from about 1 to about 25.

In some embodiments, n can be an integer from about 1 to about 25.

In some embodiments, m/(m+n) can be from about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, or about 0.9 to about 0.99, about 0.02, about 0.06, about 0.12, about 0.26, or about 0.48.

In some embodiments, m/(m+n+p) can be from about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, or about 0.9 to about 0.99, about 0.02, about 0.06, about 0.12, about 0.26, or about 0.48.

In some embodiments, the drug-loaded compound can be crosslinked with a crosslinker.

In some embodiments, the crosslinker can include a thiol moiety. In some embodiments, the crosslinker can be

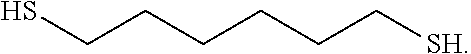

In some embodiments, the drug-loaded compound can be about 1% to about 99% crosslinked, about 1% to about 9% crosslinked, about 10% to about 19% crosslinked, about 20% to about 29% crosslinked, about 30% to about 39% crosslinked, about 40% to about 49% crosslinked, about 50% to about 59% crosslinked, about 60% to about 69% crosslinked, about 70% to about 79% crosslinked, about 80% to about 89% crosslinked, about 90% to about 99% crosslinked, about 2% crosslinked, about 6% crosslinked, about 12% crosslinked, about 26% crosslinked, or about 48% crosslinked.

In some embodiments, the drug-loaded compound retains more than 90% of its initial mass after exposure to an aqueous medium for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days at about 37° C.

In some embodiments, the aqueous medium can include PBS.

In some embodiments, the PBS includes about 0.5 wt % SDS.

In some embodiments, the drug-loaded compound can be loaded into a particle.

In some embodiments, the particle can be a nanoparticle.

In some embodiments, the particle can be a microparticle.

In some embodiments, the particle can be a macroparticle.

In some embodiments, a method of effecting controlled drug release within a subject can be employed, the method comprising administering to the subject any of the aforementioned drug-loaded compounds.

In some embodiments, the drug can be a small molecule drug.

In some embodiments, the drug can be a large molecule drug.

In some embodiments, the drug can be PTX, TAA, or TAH.

In some embodiments, the cumulative release rate of the drug from the drug-loaded compound in a an aqueous medium can be between about 10 wt % and about 20 wt % over a period of 35 days.

In some embodiments, the aqueous medium can be a solution of PBS and 0.5% SDS.

In some embodiments, the cumulative release rate of the drug from the drug-loaded compound in an aqueous medium can be between about 40 wt % and about 50 wt % over a period of 60 days.

In some embodiments, the aqueous medium can be a solution of PBS and 0.5% SDS.

With the use of compounds, methods, and drug-loaded compounds described herein, the precision of drug release can be finely tuned for a slow, controlled release. The compounds, methods, and drug-loaded compounds disclosed herein can improve drug release kinetics, and thereby improve compliance with pharmaceutical regulations related to drug delivery.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 shows the NMR data from a polymer with 12% allyl-lactide, referred to herein as PLA-12.

FIG. 2 shows degradation rates of various polymers in PBS.

FIG. 3 shows degradation rates of various polymers in PBS and 0.5% SDS.

FIG. 4 shows degradation of polymer discs over 60 days.

FIG. 5 shows release rates of PTX in various polymers in PBS and 0.5% SDS.

FIG. 6 shows release rates of TAA in various polymers in PBS and 0.5% SDS.

FIGS. 7A and 7B show PTX release rates achieved with a media change every 3-4 days due to the drug solubility limit.

### DETAILED DESCRIPTION OF THE INVENTION

The present disclosure relates in part to the discovery that crosslinking density of a polymer is directly related to the degradation rate of said polymer, and thereby the release rate of a drug from said polymer. Drug delivery is a parameter that can be finely and predictably tuned in order to deliver a pharmaceutical compound in a patient to achieve a desired therapeutic effect. In addition to the location within the patient's body, the time frame over which the pharmaceutical compound is delivered is an important parameter that can be manipulated to achieve the desired therapeutic effect. Polymers can often be combined with a drug, and the structural characteristics of the polymers can influence the release rate of the drug from the polymer. These structural characteristics can include the types of residues, the molecular weight, the degree of crosslinking, and several other parameters. Specifically, compounds or pharmaceutically acceptable salts thereof, which include allyl lactide residues, lactide residues, and are substantially free of valerolactone residue, can be beneficial in the slow delivery of a drug to a patient, as the exclusion of valerolactone residues gives way to finer control of polymer degradation. That is, the claimed compounds enable tunable, predictable degradation and drug release profiles as a function of crosslinking density. A controlled delivery of a drug can include the release of a drug over a period of at least 30 days. In some embodiments, a controlled delivery of a drug can include the release of a drug over a period of less than 30 days (e.g., with polymers including lactide and/or glycolide residues).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present disclosure, illustrative methods and materials are described.

As used herein, each of the following terms has the meaning associated with it in this section.

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of 20% or 10% (e.g., +5%; +1%; and ±0.1%) from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the terms “alkyl”, “alkenyl”, “alkynyl” and “alkoxy” include both straight chain and branched chain groups, as well as unsubstituted and substituted groups. The optional substituents may include, without limitation, halogen, C₁-C₆ alkoxy, CN, OH, NH₂, NO₂, NH(C₁-C₆ alkyl), N(C₁-C₆ alkyl)₂, CONH₂, CO(C₁-C₆ alkyl), SO₂NH₂, and SO₂(C₁-C₆ alkyl).

As used herein, the terms “co-administered” and “co-administration” refer to administering to the subject a compound contemplated herein or pharmaceutically acceptable salt thereof along with a compound that may also treat the disorders or diseases contemplated herein. In some embodiments, the co-administered compounds are administered separately as part of a single therapeutic approach. In some embodiments, the co-administered compounds are administered in any kind of combination as part of a single therapeutic approach. In some embodiments, the co-administered compound may be formulated in any kind of combinations as mixtures of solids and liquids under a variety of solid, gel, and liquid formulations, and/or as a solution.

As used herein, the term “composition” or “pharmaceutical composition” refers to a mixture of at least one compound contemplated herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary, and topical administration.

The term “container” includes any receptacle for holding the pharmaceutical composition or to add protection to manage stability and or water-uptake. For example, in some embodiments, the container is the packaging that contains the pharmaceutical composition such as liquid (solution or suspension); semisolid; lyophilized solid; or solution, powder, or lyophilized formulation present in dual chambers. In some embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. In some embodiments, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a breathing disorder in a patient.

A “disease” as used herein is a state of health of a subject wherein the subject cannot maintain homeostasis, and wherein if the disease is not ameliorated then the subject's health continues to deteriorate.

A “disorder” as used herein in a subject is a state of health in which the subject is able to maintain homeostasis, but in which the subject's state of health is less favourable than it would be in the absence of the disorder. Left untreated, a disorder does not necessarily cause a further decrease in the subject's state of health.

As used herein, the terms “effective amount,” “pharmaceutically effective amount” and “therapeutically effective amount” refer to a nontoxic but sufficient amount of a compound or agent to provide the desired biological result. That result may be reduction and/or alleviation of the signs, symptoms, or causes of a disease, or any other desired alteration of a biological system. That result may be reduction and/or alleviation of the signs or symptoms of a disease, or any other desired alteration of a biological system. An appropriate therapeutic amount in any individual case may be determined by one of ordinary skill in the art using routine experimentation. A therapeutic benefit or improvement need not be complete ablation of any one, most, or all symptoms, complications, consequences, or underlying causes associated with the disorder or disease. Thus, in some embodiments, a satisfactory endpoint is achieved when there is a transient, medium, or long term, incremental improvement in a subject's condition, or a partial reduction in the occurrence, frequency, severity, progression, or duration, or inhibition or reversal, of one or more associated adverse symptoms, complications, consequences, or underlying causes, worsening, or progression (e.g., stabilizing one or more symptoms or complications of the condition, disorder, or disease), of the disorder or disease, over a duration of time (e.g., hours, days, weeks, months, or years).

As used herein, “instructional material”, includes a publication, a recording, a diagram, or any other medium of expression that can be used to communicate the usefulness of a composition and/or compound contemplated herein in a kit. The instructional material of the kit may, for example, be affixed to a container that contains the compound and/or composition contemplated herein. The instructional material of the kit may, for example, be shipped together with a container that contains the compound and/or composition. The instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail or download from a website.

As used herein, “likelihood”, “likely to”, and similar generally refers to an increase in the probability of an event. Thus, “likelihood”, “likely to”, and similar, when used in reference to responsiveness to cancer therapy, contemplates an increased probability that the individual will exhibit a reduction in the severity of cancer or the symptoms of cancer or the retardation or slowing of the cancer progression. The term “likelihood”, “likely to”, and similar, when used in reference to responsiveness to cancer therapy, can also mean the increase of indicators that may be evidence of the cancer being responsive to treatment.

The terms “patient,” “subject” or “individual” are used interchangeably herein, and refer to any animal, or cells thereof whether in vitro or in situ, amenable to the methods described herein. In some embodiments, the subject is a patient. In some embodiments, the patient is an animal. In some embodiments, the patient is a mammal. In some embodiments, the patient is a human. In some embodiments, the subject is a subject in need of treatment thereof.

As used herein, a “particle” refers to a small portion of matter. A “nanoparticle” refers to a particle with an average dimension of between about 1 nm and about 999 nm. A “microparticle” refers to a particle with an average dimension of between about 1 μm and about 999 μm. A “macroparticle” refers to a particle with an average dimension of between about 1 mm and about 15 cm. As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound disclosed herein. As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound disclosed herein to the subject such that it may perform its intended function. In some embodiments, such constructs are carried or transported from one organ, or portion of the body, to another organ, or portion of the body. Each carrier must be “acceptable” in the sense of being compatible with the other ingredients of the formulation, including compounds disclosed herein, and not injurious to the patient. In some embodiments, examples of materials that may serve as pharmaceutically acceptable carriers include: sugars (e.g., lactose, glucose, and sucrose); starches (e.g., corn starch and potato starch); cellulose, and its derivatives (e.g., sodium carboxymethyl cellulose, ethyl cellulose, and cellulose acetate); powdered tragacanth; malt; gelatin; talc; excipients, such as cocoa butter and suppository waxes; oils (e.g., peanut oil, cottonseed oil, safflower oil, sesame oil, olive oil, corn oil, and soybean oil); glycols (e.g., propylene glycol); polyols (e.g., glycerin, sorbitol, mannitol, and polyethylene glycol, referred to hereinafter as “PEG”); esters (e.g., ethyl oleate and ethyl laurate); agar; buffering agents (e.g., magnesium hydroxide and aluminium hydroxide); surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes, for example, coatings, antibacterial and antifungal agents, and absorption delaying agents, and the like that are compatible with the activity of the compounds disclosed herein and are physiologically acceptable to the subject. Supplementary active compounds may also be incorporated into the compositions.

The “pharmaceutically acceptable carrier” may further include a pharmaceutically acceptable salt of the compounds disclosed herein. Other additional ingredients that may be included in the pharmaceutical compositions disclosed herein are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the compounds of the present disclosure prepared from, for example, pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

As used herein, the term “prodrug” refers to a compound that, after administration, is metabolised or otherwise converted to a biologically active or more active compound (e.g., drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability, altered transport characteristics, fewer side effects, lower toxicity, or improved flavour (for example, see the reference Nogrady, 1985, Medicinal Chemistry A Biochemical Approach, Oxford University Press, New York, pages 388-392, incorporated herein by reference). A prodrug may be synthesized using reactants other than the corresponding compound (e.g., drug).

A “therapeutic” treatment is a treatment administered to a subject who exhibits signs of pathology for the purpose of diminishing or eliminating those signs.

The term “responsiveness” or “responsive,” when used in reference to a treatment, refers to the degree of effectiveness of the treatment in lessening or decreasing the symptoms of a disease, disorder, or condition being treated. For example, the term “increased responsiveness,” when used in reference to a treatment of a cell or a subject, refers to an increase in the effectiveness in lessening or decreasing the symptoms of the disease when measured using any methods known in the art. In some embodiments, the increase in the effectiveness is at least about 5%, at least about 10%, at least about 15%, at least about 20%, at least about 25%, at least about 30%, at least about 35%, at least about 40%, at least about 45%, or at least about 50%.

As used herein, the term “treatment” or “treating” is defined as the application or administration of a therapeutic agent, i.e., a compound disclosed herein (alone or in combination with another pharmaceutical agent), to a subject, or application or administration of a therapeutic agent to an isolated tissue or cell line from a subject (e.g., for diagnosis or ex vivo applications), who has a condition contemplated herein, a symptom of a condition contemplated herein, or the potential to develop a condition contemplated herein, with the purpose to cure, heal, alleviate, relieve, alter, remedy, ameliorate, improve, or affect a condition contemplated herein, the symptoms of a condition contemplated herein or the potential to develop a condition contemplated herein. In some embodiments, the term “treatment” or “treating” is defined as the application or administration of a therapeutic agent, i.e., a compound disclosed herein (alone or in combination with another pharmaceutical agent), to a subject, or application or administration of a therapeutic agent to an isolated tissue or cell line from a subject (e.g., for diagnosis or ex vivo applications), who has a condition contemplated herein or a symptom of a condition contemplated herein, with the purpose to cure, heal, alleviate, relieve, alter, remedy, ameliorate, improve, or affect a condition contemplated herein, the symptoms of a condition contemplated herein or the potential to develop a condition contemplated herein. In some embodiments, the term “treatment” or “treating” is defined as the application or administration of a therapeutic agent, i.e., a compound disclosed herein (alone or in combination with another pharmaceutical agent), to a subject, or application or administration of a therapeutic agent to an isolated tissue or cell line from a subject (e.g., for diagnosis or ex vivo applications), who has a condition contemplated herein or a symptom of a condition contemplated herein, with the purpose to alleviate, relieve, alter, remedy, ameliorate, improve, or affect a condition contemplated herein, the symptoms of a condition contemplated herein or the potential to develop a condition contemplated herein. Such treatments may be specifically tailored or modified, based on knowledge obtained from the field of pharmacogenomics.

In some embodiments, the term “treatment” or “treating” refers to an action that occurs while an individual is suffering from a specified disease, which reduces the severity of the disease or the symptoms of the disease, and/or retards or slows the progression of the disease. For instance, in some embodiments, “treatment” or “treat” refers to an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% decrease in the rate of progress of a disease. In some embodiments, “treatment” refers to an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% decrease in any physical symptom(s) of a disease. In some embodiments, “treatment” refers to an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% increase in the general health of the individual, as determined by any suitable means, such as cell counts, assay results, or other suitable means.

Throughout the present disclosure, various aspects of the disclosure can be presented in a range format. It should be understood that the description in range format is merely for convenience and brevity and should not be construed as an inflexible limitation on the disclosure herein. Accordingly, the description of a range should be considered to have specifically disclosed all the possible sub-ranges as well as individual numerical values within that range. For example, description of a range such as from about 1 to about 6 should be considered to have specifically disclosed sub-ranges such as from about 1 to about 3, from about 1 to about 4, from about 1 to about 5, from about 2 to about 4, from about 2 to about 6, from about 3 to about 6, etc., as well as individual numbers within that range, for example, about 1, about 2, about 2.7, about 3, about 4, about 5, about 5.1, about 5.3, about 5.5, and about 6. Thus, for example, reference to a range of 90-100% includes about 91-99%, about 92-98%, about 93-95%, about 91-98%, about 91-97%, about 91-96%, about 91-95%, about 91-94%, about 91-93%, and so forth. Reference to a range of 90-100% also includes about 91%, about 92%, about 93%, about 94%, about 95%, about 96%, about 97%, etc., as well as about 91.1%, about 91.2%, about 91.3%, about 91.4%, about 91.5%, etc., and about 92.1%, about 92.2%, about 92.3%, about 92.4%, about 92.5%, and so forth. A series of ranges are disclosed throughout the present disclosure. The use of a series of ranges includes combinations of the upper and lower ranges to provide another range. This construction applies regardless of the breadth of the range and in all contexts throughout this present disclosure. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth. This applies regardless of the breadth of the range.

### Polymers Containing Allyl-Lactide Residues and Lactide Residues.

Embodiments described herein relate generally to compounds comprising lactide and allyl lactide residues with substantially no valerolactone residues, methods of producing said compounds, methods of loading drugs into said compounds, and drug-loaded compounds comprising lactide residues, allyl lactide residues, and substantially no valerolactone residues. One aspect described herein relates generally to a compound or a pharmaceutically acceptable salt thereof, comprising allyl lactide residues and lactide residues, wherein the compound or pharmaceutically acceptable salt thereof is substantially free of valerolactone residues. Technical advantages of a compound that includes allyl lactide residues, lactide residues, and are substantially free of valerolactone residues include, but are not limited to fine control of crosslinking density, fine control of compound degradation, and fine control of release rate control of a drug contained in the compound. Release rate of drugs from polymer is a function of crosslinking density. Compound degradation is also a function of crosslinking density. Crosslinking density can be correlated with allyl percentage, as allyl groups are often the primary sites that are reactive with crosslinkers.

In some embodiments, the compound can include glycolide residues. In some embodiments, the compound can include PEG residues. In some embodiments, the compound can include benzyl residues. In some embodiments, benzene can be used as an initiator. In some embodiments, the compound can include benzyl residues and PEG residues. In some embodiments, the compound can include glycolide and PEG residues. In some embodiments, the compound can include glycolide and benzyl residues. In some embodiments, the compound can include glycolide, PEG, and benzyl residues. In some embodiments, the compound can include no benzyl residues.

In some embodiments, a catalyst can be used in the production of the compound. In some embodiments, the catalyst can be a tin-based catalyst. In some embodiments, the catalyst can include Tin(II) trifluoromethanesulfonate. In some embodiments, the catalyst can include 4-Dimethylaminopyridine (DMAP). In some embodiments, the compound can be produced without the use of a catalyst.

In some embodiments, the compound can include about 1 wt % to about 99 wt %, about 1 wt % to about 9 wt %, about 10 wt % to about 19 wt %, about 20 wt % to about 29 wt %, about 30 wt % to about 39 wt %, about 40 wt % to about 49 wt %, about 50 wt % to about 59 wt %, about 60 wt % to about 69 wt %, about 70 wt % to about 79 wt %, about 80 wt % to about 89 wt %, about 90 wt % to about 99 wt % lactide residues, or about 100 wt % lactide residues, inclusive of all values and ranges therebetween.

In some embodiments, the compound can include about 1 wt % to about 100 wt %, about 1 wt % to about 99 wt %, about 1 wt % to about 9 wt %, about 10 wt % to about 19 wt %, about 20 wt % to about 29 wt %, about 30 wt % to about 39 wt %, about 40 wt % to about 49 wt %, about 50 wt % to about 59 wt %, about 60 wt % to about 69 wt %, about 70 wt % to about 79 wt %, about 80 wt % to about 89 wt %, about 90 wt % to about 99 wt % allyl-lactide residues, about 100 wt % allyl-lactide residues, about 2 wt % allyl-lactide residues, about 6 wt % allyl-lactide residues, about 12 wt % allyl-lactide residues, about 26 wt % allyl-lactide residues, or about 48 wt % allyl-lactide residues, inclusive of all values and ranges therebetween.

In some embodiments, the compound can include about 1 wt % to about 99 wt %, about 1 wt % to about 9 wt %, about 10 wt % to about 19 wt %, about 20 wt % to about 29 wt %, about 30 wt % to about 39 wt %, about 40 wt % to about 49 wt %, about 50 wt % to about 59 wt %, about 60 wt % to about 69 wt %, about 70 wt % to about 79 wt %, about 80 wt % to about 89 wt %, or about 90 wt % to about 99 wt % glycolide residues, inclusive of all values and ranges therebetween.

In some embodiments, the compound can include about 1 wt % to about 99 wt %, about 1 wt % to about 9 wt %, about 10 wt % to about 19 wt %, about 20 wt % to about 29 wt %, about 30 wt % to about 39 wt %, about 40 wt % to about 49 wt %, about 50 wt % to about 59 wt %, about 60 wt % to about 69 wt %, about 70 wt % to about 79 wt %, about 80 wt % to about 89 wt %, or about 90 wt % to about 99 wt % PEG residues, inclusive of all values and ranges therebetween.

In some embodiments, the compound can include about 1 wt % to about 99 wt %, about 1 wt % to about 9 wt %, about 10 wt % to about 19 wt %, about 20 wt % to about 29 wt %, about 30 wt % to about 39 wt %, about 40 wt % to about 49 wt %, about 50 wt % to about 59 wt %, about 60 wt % to about 69 wt %, about 70 wt % to about 79 wt %, about 80 wt % to about 89 wt %, or about 90 wt % to about 99 wt % benzyl residues, inclusive of all values and ranges therebetween.

In some embodiments, the compound can have the following structure:

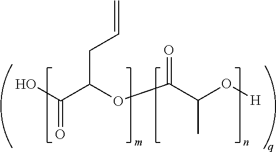

In some embodiments, m can be an integer from about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 1 to about 20, about 1 to about 15, about 5 to about 15, about 10 to about 15, about 10 to about 20, about 1 to about 10, about 5 to about 10, about 20 to about 25, about 20 to about 30, about 25 to about 30, or about 15 to about 20, inclusive of all values and ranges therebetween.

In some embodiments, n can be an integer from about 0 to about 1,000, about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 100 to about 900, about 100 to about 800, about 100 to about 700, about 100 to about 600, about 100 to about 500, about 100 to about 400, about 100 to about 300, about 100 to about 200, about 100 to about 150, about 150 to about 200, about 200 to about 250, about 200 to about 225, about 125 to about 150, about 150 to about 175, about 140 to about 150, about 135 to about 145, about 135 to about 140, about 140 to about 145, about 50 to about 75, about 50 to about 60, about 55 to about 60, about 60 to about 65, about 65 to about 70, or about 65 to about 75, inclusive of all values and ranges therebetween.

In some embodiments, q can be an integer from about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 1 to about 15, about 1 to about 10, about 9, about 8, about 7, about 6, about 5, about 4, about 3, about 2, or about 1, inclusive of all values and ranges therebetween.

In some embodiments, the compound can have the following structure:

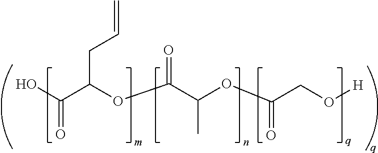

In some embodiments, m can be an integer from about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 1 to about 20, about 1 to about 15, about 5 to about 15, about 10 to about 15, about 10 to about 20, or about 15 to about 20, inclusive of all values and ranges therebetween.

In some embodiments, n can be an integer from about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 100 to about 900, about 100 to about 800, about 100 to about 700, about 100 to about 600, about 100 to about 500, about 100 to about 400, about 100 to about 300, about 100 to about 200, about 100 to about 150, about 150 to about 200, about 125 to about 150, about 150 to about 175, about 140 to about 150, about 135 to about 145, about 135 to about 140, or about 140 to about 145, inclusive of all values and ranges therebetween.

In some embodiments, p can be an integer from about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 25 to about 1,000, about 25 to about 900, about 25 to about 800, about 25 to about 700, about 25 to about 600, about 25 to about 500, about 25 to about 400, about 25 to about 300, about 25 to about 200, about 25 to about 100, about 25 to about 50, about 25 to about 40, about 30 to about 40, about 30 to about 35, or about 35 to about 40, inclusive of all values and ranges therebetween.

In some embodiments, p can be an integer from about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 1 to about 15, about 1 to about 10, about 9, about 8, about 7, about 6, about 5, about 4, about 3, about 2, about 1, inclusive of all values and ranges therebetween.

In some embodiments, q can be an integer from about 1 to about 1,000, about 1 to about 900, about 1 to about 800, about 1 to about 700, about 1 to about 600, about 1 to about 500, about 1 to about 400, about 1 to about 300, about 1 to about 200, about 1 to about 100, about 1 to about 75, about 1 to about 50, about 1 to about 25, about 1 to about 15, about 1 to about 10, about 9, about 8, about 7, about 6, about 5, about 4, about 3, about 2, about 1, inclusive of all values and ranges therebetween.

In some embodiments, m/(m+n) can be about 0.01 to about 1, about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, about 0.9 to about 0.99, inclusive of all values and ranges therebetween.

In some embodiments, m/(m+n+p) can be about 0.01 to about 1, about 0.01 to about 0.99, about 0.2 to about 0.5, about 0.4 to about 0.5, about 0.1 to about 0.19, about 0.2 to about 0.29, about 0.3 to about 0.39, about 0.4 to about 0.49, about 0.5 to about 0.59, about 0.6 to about 0.69, about 0.7 to about 0.79, about 0.8 to about 0.89, about 0.9 to about 0.99, inclusive of all values and ranges therebetween.

In some embodiments, the compound can be crosslinked. In some embodiments, the compound can include a crosslinking compound. In some embodiments, the residues of the compound can be crosslinked with a crosslinker. In some embodiments, the crosslinker can include a thiol moeity. In some embodiments, the crosslinker can have the following structure:

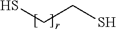

In some embodiments, f can be about 0, about 1, about 2, about 3, about 4, about 5, about 6, about 7, about 8, about 9, about 10, about 15, about 20, about 25, about 30, inclusive of all ranges and values therebetween.

In some embodiments, the crosslinker can be a cleavable crosslinker. In some embodiments, the crosslinker can include PEG.

In some embodiments the crosslinker can have the following structure:

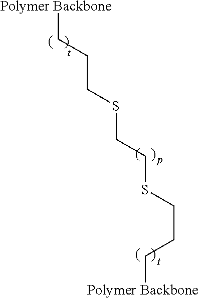

In some embodiments, polymer backbones can be joined by multiple crosslinkers.

In some embodiments, p can be an integer from about 0 to about 5. In some embodiments, t can be an integer from about 0 to about 5.

In some embodiments the crosslinker can have the following structure:

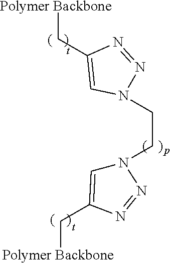

In some embodiments, p can be an integer from about 0 to about 5. In some embodiments, t can be an integer from about 0 to about 5.

In some embodiments the crosslinker can have the following structure:

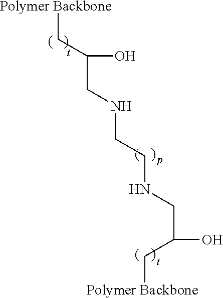

In some embodiments, p can be an integer from about 0 to about 5. In some embodiments, t can be an integer from about 0 to about 5.

In some embodiments the crosslinker can have the following structure:

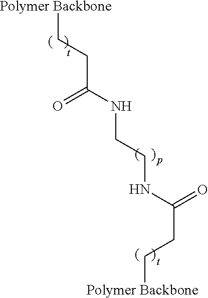

In some embodiments, p can be an integer from about 0 to about 5. In some embodiments, t can be an integer from about 0 to about 5.

In some embodiments the crosslinker can have the following structure:

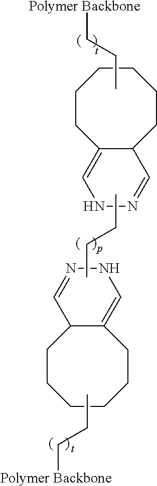

In some embodiments, p can be an integer from about 0 to about 5. In some embodiments, t can be an integer from about 0 to about 5.

In some embodiments the crosslinker can have the following structure:

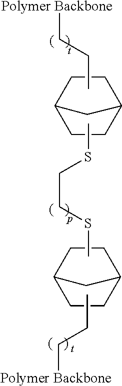

In some embodiments, p can be an integer from about 0 to about 5. In some embodiments, t can be an integer from about 0 to about 5.

In some embodiments the crosslinker can have the following structure:

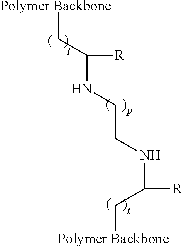

In some embodiments, p can be an integer from about 0 to about 5. In some embodiments, t can be an integer from about 0 to about 5.

In some embodiments, the compound can be about 1% to about 100% crosslinked, about 1% to about 99% crosslinked, about 1% to about 9% crosslinked, about 10% to about 19% crosslinked, about 20% to about 29% crosslinked, about 30% to about 39% crosslinked, about 40% to about 49% crosslinked, about 50% to about 59% crosslinked, about 60% to about 69% crosslinked, about 70% to about 79% crosslinked, about 80% to about 89% crosslinked, about 90% to about 99% crosslinked, about 100% crosslinked, about 2% crosslinked, about 6% crosslinked, about 12% crosslinked, about 26% crosslinked, or about 48% crosslinked, inclusive of all values and ranges therebetween.

In some embodiments, the compound can have a weight average molecular weight (Mw) of about 5 kDa to about 50 kDa, about 5 kDa to about 45 kDa, about 5 kDa to about 40 kDa, about 5 kDa to about 35 kDa, about 5 kDa to about 30 kDa, about 5 kDa to about 25 kDa, about 5 kDa to about 20 kDa, about 5 kDa to about 15 kDa, about 5 kDa to about 10 kDa, about 10 kDa to about 50 kDa, about 10 kDa to about 45 kDa, about 10 kDa to about 40 kDa, about 10 kDa to about 35 kDa, about 10 kDa to about 30 kDa, about 10 kDa to about 25 kDa, about 10 kDa to about 20 kDa, about 10 kDa to about 15 kDa, about 15 kDa to about 50 kDa, about 15 kDa to about 45 kDa, about 15 kDa to about 35 kDa, about 15 kDa to about 30 kDa, about 15 kDa to about 25 kDa, about 15 kDa to about 20 kDa, about 20 kDa to about 50 kDa, about 20 kDa to about 45 kDa, about 20 kDa to about 40 kDa, about 20 kDa to about 35 kDa, about 20 kDa to about 30 kDa, about 20 kDa to about 25 kDa, about 25 kDa to about 50 kDa, about 25 kDa to about 45 kDa, about 25 kDa to about 40 kDa, about 25 kDa to about 35 kDa, about 25 kDa to about 30 kDa, about 30 kDa to about 50 kDa, about 30 kDa to about 45 kDa, about 30 kDa to about 40 kDa, about 30 kDa to about 35 kDa, about 35 kDa to about 50 kDa, about 35 kDa to about 45 kDa, about 35 kDa to about 40 kDa, about 40 kDa to about 50 kDa, about 40 kDa to about 45 kDa, or about 45 kDa to about 50 kDa, inclusive of all values and ranges therebetween.

In some embodiments, the compound can have a number average molecular weight (Mn) of about 5 kDa to about 50 kDa, about 5 kDa to about 45 kDa, about 5 kDa to about 40 kDa, about 5 kDa to about 35 kDa, about 5 kDa to about 30 kDa, about 5 kDa to about 25 kDa, about 5 kDa to about 20 kDa, about 5 kDa to about 15 kDa, about 5 kDa to about 10 kDa, about 10 kDa to about 50 kDa, about 10 kDa to about 45 kDa, about 10 kDa to about 40 kDa, about 10 kDa to about 35 kDa, about 10 kDa to about 30 kDa, about 10 kDa to about 25 kDa, about 10 kDa to about 20 kDa, about 10 kDa to about 15 kDa, about 15 kDa to about 50 kDa, about 15 kDa to about 45 kDa, about 15 kDa to about 35 kDa, about 15 kDa to about 30 kDa, about 15 kDa to about 25 kDa, about 15 kDa to about 20 kDa, about 20 kDa to about 50 kDa, about 20 kDa to about 45 kDa, about 20 kDa to about 40 kDa, about 20 kDa to about 35 kDa, about 20 kDa to about 30 kDa, about 20 kDa to about 25 kDa, about 25 kDa to about 50 kDa, about 25 kDa to about 45 kDa, about 25 kDa to about 40 kDa, about 25 kDa to about 35 kDa, about 25 kDa to about 30 kDa, about 30 kDa to about 50 kDa, about 30 kDa to about 45 kDa, about 30 kDa to about 40 kDa, about 30 kDa to about 35 kDa, about 35 kDa to about 50 kDa, about 35 kDa to about 45 kDa, about 35 kDa to about 40 kDa, about 40 kDa to about 50 kDa, about 40 kDa to about 45 kDa, or about 45 kDa to about 50 kDa, inclusive of all values and ranges therebetween.

In some embodiments, the compound can have a polydispersity index (PDI) if about 1 to about 2, about 1 to about 1.9, about 1 to about 1.8, about 1 to about 1.7, about 1 to about 1.6, about 1 to about 1.5, about 1 to about 1.4, about 1 to about 1.3, about 1 to about 1.2, about 1 to about 1.1, about 1.1 to about 2, about 1.1 to about 1.9, about 1.1 to about 1.8, about 1.1 to about 1.7, about 1.1 to about 1.6, about 1.1 to about 1.5, about 1.1 to about 1.4, about 1.1 to about 1.3, about 1.1 to about 1.2, about 1.2 to about 2, about 1.2 to about 1.9, about 1.2 to about 1.8, about 1.2 to about 1.7, about 1.2 to about 1.6, about 1.2 to about 1.5, about 1.2 to about 1.4, about 1.2 to about 1.3, about 1.3 to about 2, about 1.3 to about 1.9, about 1.3 to about 1.8, about 1.3 to about 1.7, about 1.3 to about 1.6, about 1.3 to about 1.5, about 1.3 to about 1.4, about 1.4 to about 2, about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2. about 1.8 to about 1.9, or about 1.9 to about 2, inclusive of all values and ranges therebetween.

In some embodiments, the compound retains more than 90% of its initial mass after exposure to a PBS for an exposure period at about 37° C. In some embodiments, the compound retains more than 99%, more than 98%, more than 97%, more than 96%, more than 95%, more than 94%, more than 93%, more than 92%, more than 91%, more than 90%, more than 85%, more than 80%, more than 75%, more than 70%, more than 65%, more than 60%, more than 55%, or more than 50% of its initial mass after exposure to a PBS for 20 days at about 37° C., inclusive of all values and ranges therebetween. In some embodiments, the PBS can include about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt %, about 9 wt %, or about 10 wt % SDS, inclusive of all values and ranges therebetween.

In some embodiments, the exposure period can be about 1 day, about 2 days, about 3 days, about 4 days, about 5 days, about 6 days, about 7 days, about 8 days, about 9 days, about 10 days, 11 days, 12 days, 13 days, 14 days, about 15 days, about 16 days, about 17 days, about 18 days, about 19 days, about 20 days, about 25 days, about 30 days, about 35 days, about 40 days, about 45 days, about 50 days, about 55 days, or about 60 days, inclusive of all values and ranges therebetween.

In some embodiments, the compound can be included in a particle. In some embodiments, the compound can be included in a nanoparticle. In some embodiments, the compound can be included in a microparticle. In some embodiments, the compound can be included in a microparticle. In some embodiments, the particle can have an average dimension of about 1 nm to about 500 nm, about 1 nm to about 400 nm, about 1 nm to about 300 nm, about 1 nm to about 200 nm, about 1 nm to about 100 nm, about 1 nm to about 50 nm, about 1 μm to about 500 μm, about 1 μm to about 400 μm, about 1 μm to about 300 μm, about 1 μm to about 200 μm, about 1 μm to about 100 μm, about 1 μm to about 50 μm, about 1 μm to about 40 μm, about 1 μm to about 30 μm, about 1 μm to about 20 μm, about 1 μm to about 10 μm, about 1 μm to about 5 μm, about 1 μm to about 4 μm, about 1 μm to about 3 μm, about 1 μm to about 2 μm, about 500 μm to about 5 mm, about 500 μm to about 4 mm, about 500 μm to about 3 mm, about 500 μm to about 2 mm, about 500 μm to about 1 mm, about 1 mm to about 5 mm, about 1 mm to about 4 mm, about 1 mm to about 3 mm, or about 1 mm to about 2 mm, inclusive of all values and ranges therebetween.

In some embodiments, the compound can be included in a macroparticle. In some embodiments, the macroparticle can be a disc. In some embodiments, the macroparticle can be a depot. In some embodiments, the macroparticle can be an implant. In some embodiments, the macroparticle can have an average dimension of about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 1 cm, about 1 mm to about 2 cm, about 1 mm to about 3 cm, about 1 mm to about 4 cm, about 1 mm to about 5 cm, about 1 mm to about 6 cm, about 1 mm to about 7 cm, about 1 mm to about 8 cm, about 1 mm to about 9 cm, about 1 mm to about 10 cm, about 1 mm to about 11 cm, about 1 mm to about 12 cm, about 1 mm to about 13 cm, about 1 mm to about 14 cm, or about 1 mm to about 15 cm, inclusive of all values and ranges therebetween.

In some embodiments, a drug can be incorporated into the compound. In some embodiments, the incorporation of the drug into the compound can include:

(i) providing the compound or a pharmaceutically acceptable salt thereof;
(ii) incubating the compound and the drug in the presence of a solvent for an incubation period to form a drug-loaded compound; and (iii) separating the drug-loaded compound from the solvent.

In some embodiments, the solvent can include a hydrophobic solvent. In some embodiments, the solvent can include a hydrophilic solvent. In some embodiments, the solvent can include a hydrophobic solvent. In some embodiments, the solvent can include a hydrophobic solvent. In some embodiments, the solvent can include a hydrophilic solvent. In some embodiments, the solvent can include a hydrophilic solvent. In some embodiments, the solvent can include dichloromethane. In some embodiments, the solvent can include water. In some embodiments, the solvent can include ethanol. In some embodiments, the solvent can include methanol. In some embodiments, the solvent can include acetic acid. In some embodiments, the solvent can include propanol. In some embodiments, the solvent can include carbon tetrachloride. In some embodiments, the solvent can include dimethyl chloride. In some embodiments, the solvent can include hexane. In some embodiments, the solvent can include benzene. In some embodiments, the solvent can include THF. In some embodiments, the solvent can include DMSO. In some embodiments, the solvent can include THF and DMSO. In some embodiments, the solvent can be about a 50:50 wt % mixture of THF:DMSO. In some embodiments, the solvent can be about a 10:90 wt %, about a 20:80 wt %, about a 30:70 wt %, about a 40:60 wt %, about a 60:40 wt %, about a 70:30 wt %, about a 80:20 wt %, about a 90:10 wt % mixture of THF:DMSO.

In some embodiments, the incubation period can be about 1 day to about 30 days, about 1 day to about 25 days, about 1 day to about 20 days, about 1 day to about 15 days, about 1 day to about 10 days, about 1 day to about 5 days, about 5 days to about 30 days, about 5 days to about 25 days, about 5 days to about 20 days, about 5 days to about 15 days, about 5 days to about 10 days, about 10 days to about 30 days, about 10 days to about 25 days, about 10 days to about 20 days, about 10 days to about 15 days, about 15 days to about 20 days, about 20 days to about 30 days, about 20 days to about 25 days, or about 25 days to about 30 days, about 1 day, about 2 days, about 4 days, about 5 days, about 6 days, about 7 days, about 8 days, about 9 days, or about 10 days, inclusive of all values and ranges therebetween.

In some embodiments, the incubation period can be at a temperature of about 20° C. to about 50° C., about 20° C. to about 40° C., about 20° C. to about 30° C., about 30° C. to about 50° C., about 30° C. to about 40° C., or about 40° C. to about 50° C.

In some embodiments, the drug can be a small molecule drug. In some embodiments, the drug can be a large molecule drug. In some embodiments, the drug can include PTX, TAA, or TAH.

In some embodiments, separating the drug-loaded compound from the solvent can include vacuum filtration, centrifugal filtration, gravity filtration, cold filtration, hot filtration, multilayer filtration, or any other suitable separation process or combination of processes.

In some embodiments, the drug-loaded compound can include about 10 wt % to about 60 wt %, about 10 wt % to about 50 wt %, about 10 wt % to about 40 wt %, about 10 wt % to about 30 wt %, about 10 wt % to about 20 wt %, about 20 wt % to about 60 wt %, about 20 wt % to about 50 wt %, about 20 wt % to about 40 wt %, about 20 wt % to about 30 wt %, about 30 wt % to about 60 wt %, about 30 wt % to about 50 wt %, about 30 wt % to about 40 wt %, about 40 wt % to about 60 wt %, about 40 wt % to about 50 wt %, or about 50 wt % to about 60 wt % of the drug.

In some embodiments, the drug-loaded compound releases no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85%, no more than about 90%, or no more than about 95% of the drug after a release period in a solution at a temperature of about 37° C.

In some embodiments, the solution can include PBS with about 0 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt %, about 9 wt %, or about 10 wt % SDS.

In some embodiments, the release period can be about 1 day, about 2 days, about 3 days, about 4 days, about 5 days, about 6 days, about 7 days, about 8 days, about 9 days, about 10 days, about 15 days, about 20 days, about 25 days, about 30 days, about 35 days, about 40 days, about 45 days, about 50 days, about 55 days, or about 60 days, inclusive of all values and ranges therebetween.

In some embodiments, the drug-loaded compound retains more than 90% of its initial mass after exposure to an aqueous medium for an exposure period at about 37° C. In some embodiments, the compound retains more than 99%, more than 98%, more than 97%, more than 96%, more than 95%, more than 94%, more than 93%, more than 92%, more than 91%, more than 90%, more than 85%, more than 80%, more than 75%, more than 70%, more than 65%, more than 60%, more than 55%, or more than 50% of its initial mass after exposure to an aqueous medium for 20 days at about 37° C., inclusive of all values and ranges therebetween.

In some embodiments, the aqueous medium can include PBS. In some embodiments, the PBS can include about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt %, about 9 wt %, or about 10 wt % SDS, inclusive of all values and ranges therebetween.

In some embodiments, the exposure period can be about 1 day, about 2 days, about 3 days, about 4 days, about 5 days, about 6 days, about 7 days, about 8 days, about 9 days, about 10 days, about 15 days, about 20 days, about 25 days, about 30 days, about 35 days, about 40 days, about 45 days, about 50 days, about 55 days, or about 60 days, inclusive of all values and ranges therebetween.

Those skilled in the art will recognize, or be able to ascertain using no more than routine experimentation, numerous equivalents to the specific procedures, embodiments, claims, and examples described herein. Such equivalents were considered to be within the scope of the present disclosure and covered by the claims appended hereto. For example, it should be understood, that modifications in reaction conditions, including, but not limited to, reaction times, reaction size/volume, and experimental reagents, such as solvents, catalysts, pressures, atmospheric conditions (e.g., nitrogen atmosphere), and reducing/oxidizing agents, with art-recognized alternatives and using no more than routine experimentation, are within the scope of the present disclosure.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present disclosure.

The following examples further illustrate aspects of the present disclosure. However, they are in no way a limitation of the teachings or disclosure of the present disclosure as set forth herein. As set forth in the Examples below, the disclosed compounds (e.g., compounds comprising lactide, allyl lactide, and optionally glycolide) exhibited tunable degradation and tunable drug release profiles such as slower drug release profiles compared with compounds that comprises valerolactone (e.g., allylvalerolactone) in the polymer backbone. Without wishing to be bound by theory, excluding valerolactone from the polymer backbone can enable greater control over the crosslinking density of the resulting compound, which in turn can enable greater control over degradation and drug release characteristics.

### EXAMPLES

The following is a non-limiting set of examples of compounds synthesized with methods described herein. Compounds that include valerolactone residues are presented in the examples only as a means of comparison with the compounds that do not include valerolactone residues, and are not to be construed as being included in the claimed subject matter.

### Abbreviations

AVL allyl-valerolactone

Da, kDa Dalton, Kilodalton

DCM Dichloromethane

EtOH Ethanol

NMR, H NMR, C NMR Nuclear Magnetic Resonance, Proton Nuclear Magnetic Resonance, Carbon-13 Nuclear Magnetic Resonance

PBS phosphate-buffer saline

PLA polylactic acid

PLGA poly(allyl-lactic-lactic-co-glycolic acid)

V-PLGA poly (allyl-δ-vlaerolactone-co-lactic-co-glycolic acid)

PTX Paclitaxel

PVL-AL Poly(allyl-lactic-co-valerolactone)

SDS sodium dodecyl sulfate

Sn(OTf)₂ Tin(II) trifluoromethanesulfonate

TAA Triamcinolone acetonide

TAH Triamcinolone hexacetonide

THF Tetrahydrofuran

### Example 1. Synthesis of Polymer with 12% Allyl-Lactide, Referred to Herein as PLA-12

A polymer with the following structure was synthesized

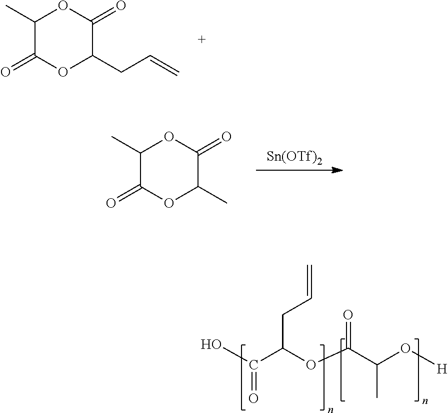

A stock solution of anhydrous ethanol in anhydrous methylene chloride was prepared (1.0 ml ethanol into 19 ml of CH2CI2, 1:20 dilution) in a 25 mL flame dried and argon-purged round bottom flask. A flame dried 50 mL round bottom flask was equipped with a stir bar, sealed with rubber septum and argon purged for 10 minutes. Then 3.6 ml of anhydrous methylene chloride was added into the reaction flask via syringe under argon to offset the created pressure. 1.24 ml of the stock ethanol solution (1.06 mmol) was added into the reaction flask via syringe under argon.

The initiator/catalyst solution was stirred at room temperature for 30 minutes. To the stirring solution, L-lactide (37.15 g) in DCM and benzyl alcohol (0.33 mL) were added via syringe, followed by addition of allyl-lactide (13.03 g) via syringe under argon balloon to offset pressure. The reaction system was stirred vigorously for 48 hours at room temperature under argon. 4-Dimethylaminopyridine (1.57 g) was weighed into to anhydrous DCM.

The resulting polymer (almost solidified) was diluted with 5 mL of methylene chloride and purified by dropwise addition into 100 ml of chilled methanol and chilled overnight in a freezer. The precipitate was rinsed with ice-cold methanol (100 ml×3) and collected by centrifuge. After overnight drying by vacuum, the final polymer product was made as a white waxy solid. EtOH to obtain a colorless crystal (95% yield). ¹H NMR (500 MHz, CDCb/TMS, ppm) δ: 5.2 (lactide), 5.7 (allyl-lactide), 7.4 (benzyl alcohol).

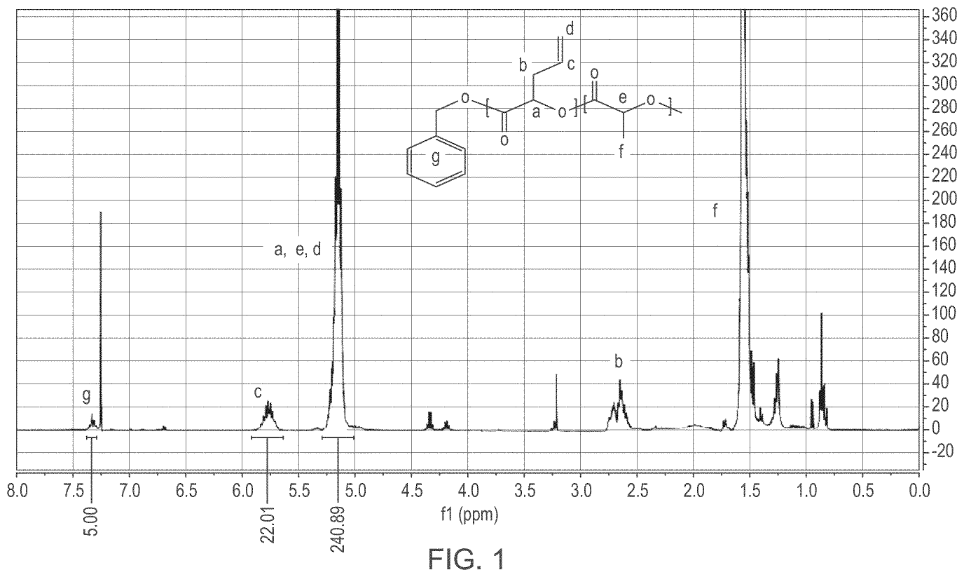

FIG. 1 shows the NMR data from Example 1.

### Example 2. Synthesis of Polymer with 6% Allyl-Lactide, Referred to Herein as PLA-6

A polymer with the following structure was synthesized:

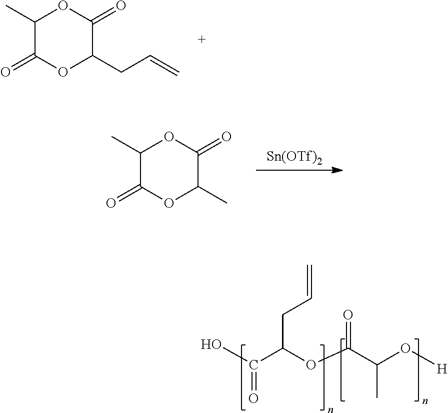

A stock solution of anhydrous ethanol in anhydrous methylene chloride was prepared (1.0 ml ethanol into 19 ml of CH2Cl2, 1:20 dilution) in a 25 mL flame dried and argon-purged round bottom flask. A flame dried 50 mL round bottom flask was equipped with a stir bar, sealed with rubber septum and argon purged for 10 minutes. Then 3.6 ml of anhydrous methylene chloride was added into the reaction flask via syringe under argon to offset the created pressure. 1.24 ml of the stock ethanol solution (1.06 mmol) was added into the reaction flask via syringe under argon.

The initiator/catalyst solution was stirred at room temperature for 30 minutes. To the stirring solution, L-lactide (37.15 g) in DCM and benzyl alcohol (0.33 mL) were added via syringe, followed by addition of allyl-lactide (13.03 g) via syringe under argon balloon to offset pressure. The reaction system was stirred vigorously for 48 hours at room temperature under argon. 4-Dimethylaminopyridine (1.46 g) was weighed into to anhydrous DCM.

The resulting polymer (almost solidified) was diluted with 5 mL of methylene chloride and purified by dropwise addition into 100 ml of chilled methanol and chilled overnight in a freezer. The precipitate was rinsed with ice-cold methanol (100 ml×3) and collected by centrifuge. After overnight drying by vacuum, the final polymer product was made as a white waxy solid. EtOH to obtain a colorless crystal (95% yield). ¹H NMR (500 MHz, CDCb/TMS, ppm) δ: 5.2 (lactide), 5.7 (allyl-lactide), 7.4 (benzyl alcohol).

### Example 3. Synthesis of Poly(Allyl-Lactic-Lactic-Co-Glycolic Acid), Referred to Herein as PLGA

A polymer with the following structure was synthesized:

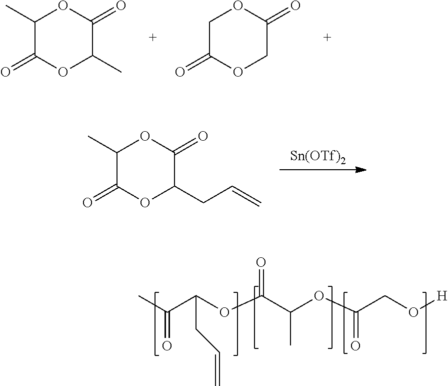

A stock solution of anhydrous ethanol in anhydrous methylene chloride was prepared (1.0 ml ethanol into 19 ml of CH2CI2, 1:20 dilution) in a 25 mL flame dried and argon-purged round bottom flask. A flame dried 50 mL round bottom flask was equipped with a stir bar, sealed with rubber septum and argon purged for 10 minutes. Sn(OTf)2 (8.2 mL, 10% weight/volume solution with toluene) was directly added into the flask. The reaction flask was capped with septum again and purged once more with argon for 10 minutes. Then 3.6 ml of anhydrous methylene chloride was added into the reaction flask via syringe under argon to offset the created pressure. 1.24 ml of the stock ethanol solution (1.06 mmol) was added into the reaction flask via syringe under argon.

The initiator/catalyst solution was stirred at room temperature for 30 minutes. To the stirring solution, allyl-lactide (8.3 g) was added via syringe, followed by addition of D,L-lactic acid (32.737 g) via syringe and a 50:50 lactic acid:glycolic acid premix (18.57 g) under argon balloon to offset pressure. The reaction system was stirred vigorously for 48 hours at room temperature under argon.

The resulting polymer (almost solidified) was diluted with 5 mL of methylene chloride and purified by dropwise addition into 100 ml of chilled methanol and chilled overnight in a freezer. The precipitate was rinsed with ice-cold methanol (100 ml×3) and collected by centrifuge. After overnight drying by vacuum, the final polymer product was made as a white waxy solid. Yield: 7.90 g/90.0%, (98.8% pure/NMR). NMR data analysis shows the polymer molecular weight at 14.9 KDa and allyl-lactide content at 13.3% by molar. NMR (500 MHz, CDCb/TMS, ppm) δ: 4.8 (glycolide), 5.2 (lactide), 5.7 (allyl-lactide).

### Example 4. Synthesis of Drug-Loaded Polymers

The polymer synthesized in Example 1 was loaded with Paclitaxel (PTX), Triamcinolone acetonide (TAA), or Triamcinolone hexacetonide (TAH) via the following process.

Drug loading was done into preformed, crosslinked microparticles and discs soaking in a prepared drug solution in either tetrahydrofuran or dichloromethane for as little as 4 hours. Paclitaxel, for example, was loaded into crosslinked microparticles. 30 mg of paclitaxel was dissolved in 0.5 mL of THF to make a drug solution, which was then used for suspending purified microparticles (16±0.5 mg) in a glass tube overnight to achieve post-drug loading via swelling and equilibrium approach. THF was then evaporated in fume hood at room temperature followed by freeze drying of paclitaxel-loaded microparticles. The resulting mixture was vortexed with 10 mL of PBS, sonicated for 1 min (until microparticles were well suspended) and centrifuged for 1 min. After the supernatant was decanted, the microparticles were retained, 10 mL of fresh PBS was replaced, vortexed without sonication, and centrifuged for 1 min. Microparticles were then freeze-dried overnight prior to release study after the decant of supernatant.

### Comparative Example 1. Synthesis of Poly(Allyl-δ-Valerolactone-Co-Lactic-Co-Glycolic Acid), Referred to Herein as AV-PLGA

A polymer with the following structure was synthesized:

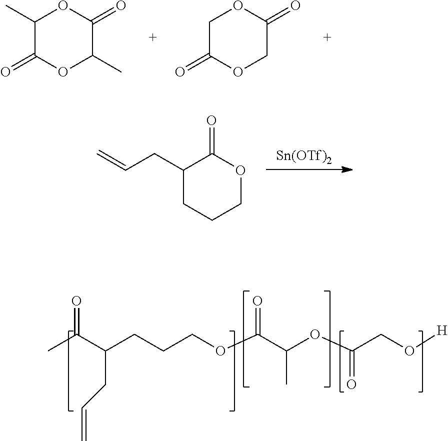

A stock solution of anhydrous ethanol in anhydrous methylene chloride was prepared (1.0 ml ethanol into 19 ml of CH2CI2, 1:20 dilution) in a 25 mL flame dried and argon-purged round bottom flask. A flame dried 50 mL round bottom flask was equipped with a stir bar, sealed with rubber septum and argon purged for 10 minutes. Sn(OTf)₂ (98.6 mg, 0.237 mmol) was directly added into the flask. The reaction flask was capped with septum again and purged once more with argon for 10 minutes. Then 3.6 ml of anhydrous methylene chloride was added into the reaction flask via syringe under argon to offset the created pressure. 1.24 ml of the stock ethanol solution (1.06 mmol) was added into the reaction flask via syringe under argon.

The initiator/catalyst solution was stirred at room temperature for 30 minutes. To the stirring solution, δ-allyl(valerolactone) (2.1 ml, 16.2 mmol) was added via syringe, followed by addition of L-lactic acid (6.05 ml, 65.3 mmol) via syringe and glycolic acid (6.05 ml, 65.3 mmol) under argon balloon to offset pressure. The reaction system was stirred vigorously for 48 hours at room temperature under argon.

The resulting polymer (almost solidified) was diluted with 5 mL of methylene chloride and purified by dropwise addition into 100 ml of chilled methanol and chilled overnight in a freezer. The precipitate was rinsed with ice-cold methanol (100 ml×3) and collected by centrifuge. After overnight drying by vacuum, the final polymer product was made as a white waxy solid. Yield: 7.90 g/90.0%, (98.8% pure/NMR). NMR data analysis shows the polymer molecular weight at 14.9 KDa and avl content at 13.3% by molar. ¹H NMR (500 MHz, CDCb/TMS, ppm) δ: 4.8 (glycolide), 5.2 (lactide), 5.7 (allyl-valerolactone).

### Comparative Example 2. Synthesis of Poly(Allyl-Lactic-Co-Valerolactone), Referred to Herein as PVL-AL

A PVL-AL polymer was synthesized and purified. NMR data analysis shows the polymer molecular weight at 14.9 KDa and avl content at 13.3% by molar. NMR (500 MHz, CDCb/TMS, ppm) δ: 4.2 (valerolactone), 5.7 (allyl-lactide).

### Example 5: Degradation in PBS and PBS with 0.5 wt % Sodium Dodecyl Sulfate (SDS)

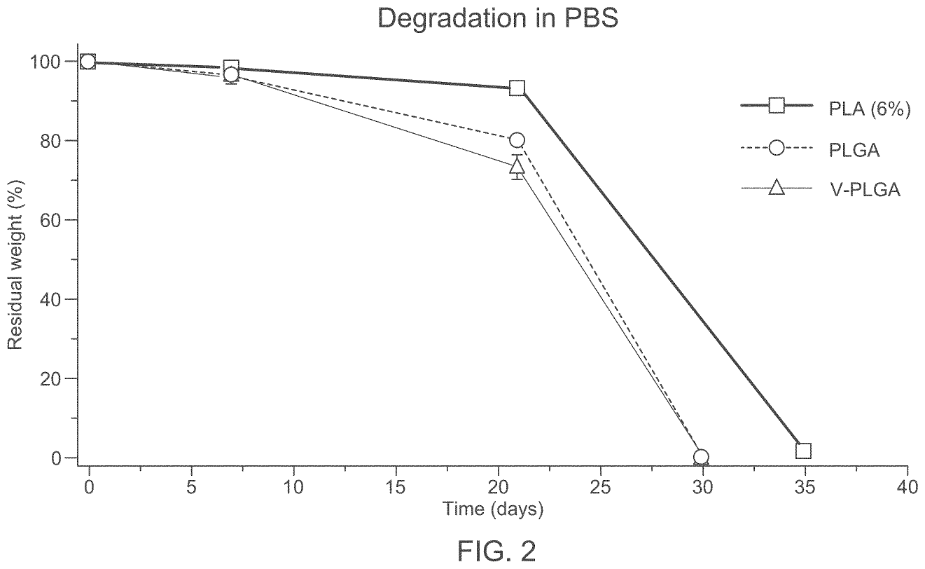

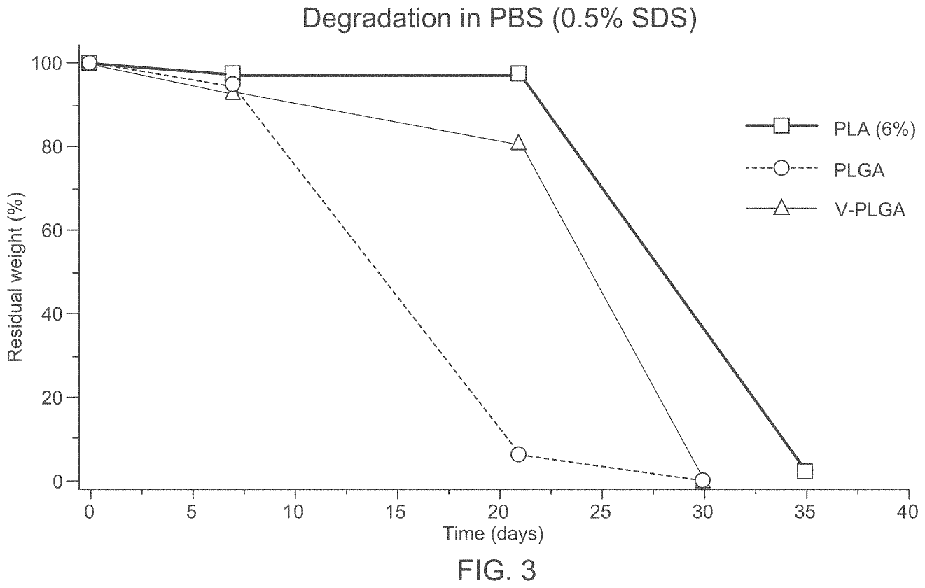

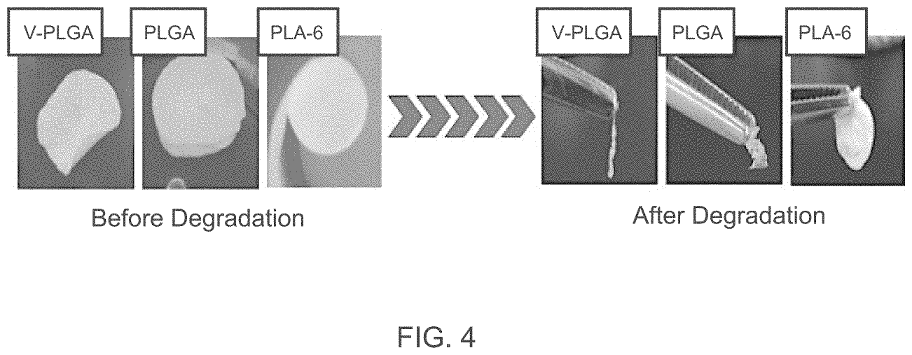

The aforementioned PLA-6, PLGA, and V-PLGA were placed in solutions of PBS and PBS with 0.5 wt % SDS and allowed to sit for 25 days. FIG. 2 and FIG. 3 present their residual weight percentages, as measured at various points throughout the process. FIG. 4 shows disc degradation over 60 days. After a period of 20 days in PBS, PLA-6 retains approximately 98% of its initial mass, PLGA retains approximately 82% of its initial mass, and V-PLGA retains approximately 74% of its initial mass. After a period of 21 days in PBS with 0.5 wt % SDS, PLA-6 retains approximately 99% of its initial mass, PLGA retains approximately 82% of its initial mass, and V-PLGA retains approximately 9% of its initial mass.

### Example 6: Release Rate of Drugs in PBS and 0.5 wt % SDS

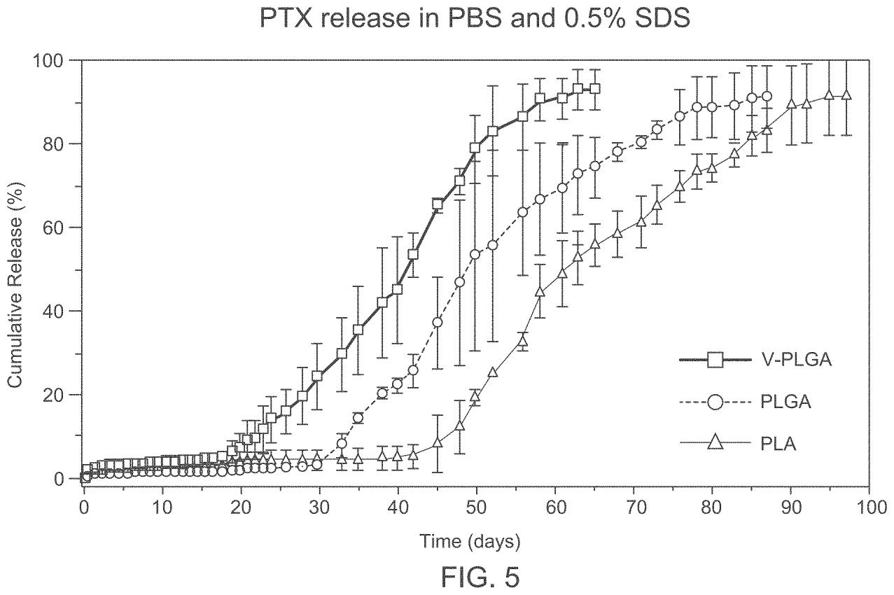

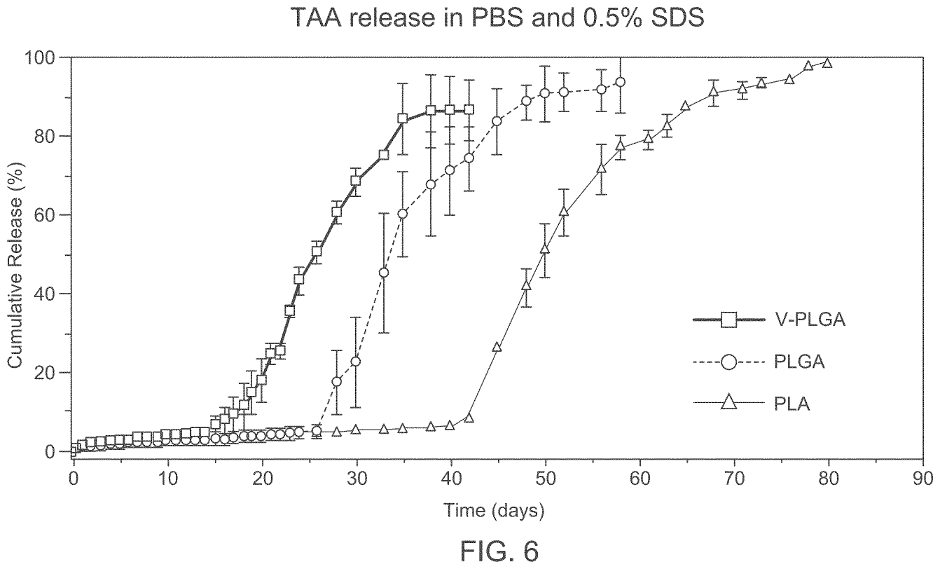

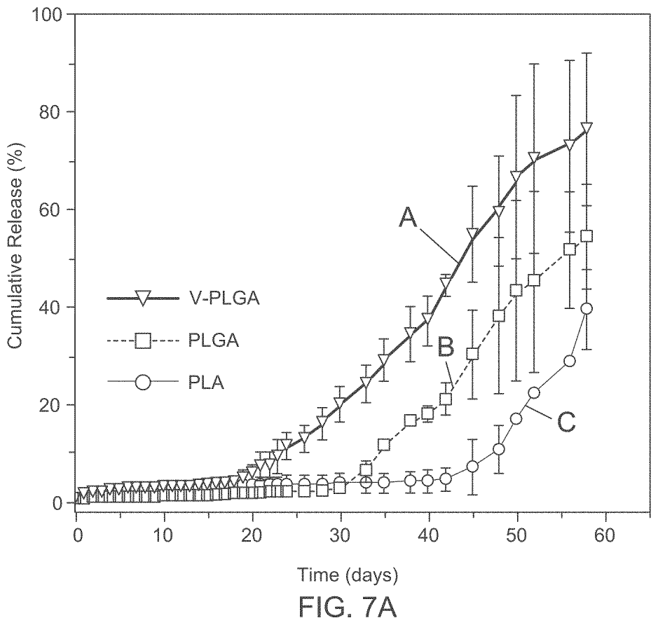

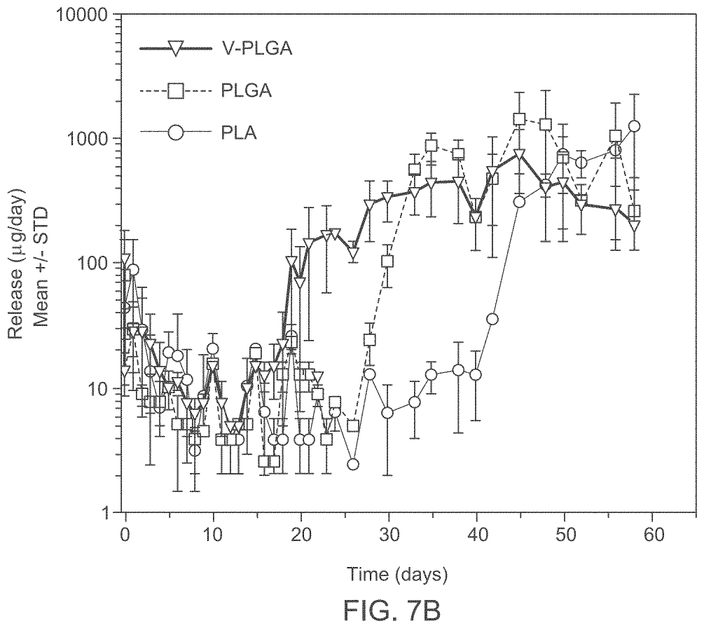

Discs of V-PLGA, PLGA, and PLA-6 were loaded with drugs. PTX (pactlictaxel) content of the loaded discs ranged from 33.81 wt % to 37.79 wt %. TAA (triamcinolone acetonide) content of the loaded discs ranged from 32.95 wt % to 35.02 wt %. Loaded discs were allowed to sit for 100 days. The following cumulative drug release rates were observed. FIG. 5 shows release rates of PTX in these polymers in PBS and 0.5% SDS. FIG. 6 shows release rates of TAA in these polymers in PBS and 0.5% SDS. FIG. 7A and FIG. 7B show PTX release rates achieved with a media change every 3-4 days due to the drug solubility limit. After a period of 60 days in PBS with 0.5 wt % SDS, PLA-6 releases approximately 50% of its initial mass of PTX, PLGA releases approximately 60% of its initial mass of PTX, and V-PLGA releases approximately 90% of its initial mass of PTX. PLA-6 releases approximately 50% of its initial mass of TAA after approximately 50 days in PBS with 0.5 wt % SDS. PLGA releases approximately 50% of its initial mass of TAA after approximately 32 days in PBS with 0.5 wt % SDS. V-PLGA releases approximately 50% of its initial mass of TAA after approximately 23 days in PBS with 0.5 wt % SDS. After 50 days with a media change every 3-4 days due to the drug solubility limit, PLA-6 releases approximately 15% of its initial mass of PTX, PLGA releases approximately 40% of its initial mass of PTX, and V-PLGA releases approximately 65% of its initial mass of PTX.

### EQUIVALENTS

The details of one or more embodiments of the disclosure are set forth in the accompanying description above. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound or a pharmaceutically acceptable salt thereof, comprising:
allyl lactide residues; and
lactide residues;
wherein the compound or pharmaceutically acceptable salt thereof is substantially free of valerolactone residues.

2. The compound of claim 1, further comprising glycolide residues.

3. The compound of claim 1, comprising the structure: 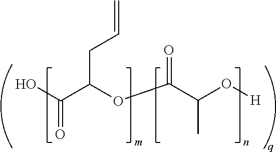
wherein:
m is an integer from about 1 to about 1,000;
n is an integer from about 1 to about 1,000;
q is an integer from about 1 to about 100; and
wherein the compound or pharmaceutically acceptable salt thereof is substantially free of valerolactone residues.

4. The compound of claim 3, having the structure: 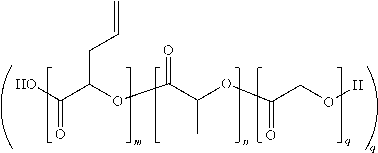
wherein
p is an integer from about 1 to about 1,000.

5. The compound of claim 3, wherein m is an integer from about 1 to about 100.

6. The compound of claim 3, wherein m is an integer from about 1 to about 25.

7. The compound of claim 3, wherein n is an integer from about 1 to about 100.

8. The compound of claim 3, wherein n is an integer from about 1 to about 25.

9. (canceled)

10. The compound of claim 3, wherein m/(m+n) is about 0.2 to about 0.5.

11. The compound of claim 3, wherein m/(m+n) is about 0.4 to about 0.5.

12-40. (canceled)

41. The compound of claim 1, wherein the residues are crosslinked with a crosslinker.

42. The compound of claim 41, wherein the crosslinker comprises a thiol moiety.

43. The compound of claim 42, wherein the crosslinker is

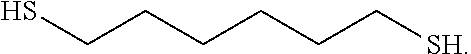

44. (canceled)

45. The compound of claim 41, wherein the compound is about 1% to about 9% crosslinked.

46. The compound of claim 41, wherein the compound is about 10% to about 19% crosslinked.

47. (canceled)

48. (canceled)

49. The compound of claim 41, wherein the compound is about 40% to about 49% crosslinked.

50-58. (canceled)

59. The compound of claim 1, wherein the compound retains more than 90% of its initial mass after exposure to an aqueous medium for 1 day at about 37° C.

60-80. (canceled)

81. A macroparticle, comprising the compound of claim 1.

82. A microparticle, comprising the compound of claim 1.

83. A nanoparticle, comprising the compound of claim 1.
